(19) United States
(12) Patent Application Publication
(10) Pub. No.: US 2005/0281856 A1
(43) Pub. Date: Dec. 22, 2005

(54) Implantable biostructure comprising an osteoconductive member and an osteoinductive material

(76) Inventors: Jonathan McGlohorn, Chapin, SC (US); Sunil Saini, Plainsboro, NJ (US); Andrea B. Caruso, Long Branch, NJ (US); Thomas George West, Lawrenceville, NJ (US); Peter A. Materna, Metuchen, NJ (US); John Sharobiem, Freehold, NJ (US); Thomas J. Bradbury, Yardley, PA (US)

Correspondence Address: HUNTON & WILLIAMS LLP;INTELLECTUAL PROPERTY DEPARTMENT, 1900 K STREET, N.W., SUITE 1200, WASHINGTON, DC, 20006-1109, US

(21) Appl. No.: 11/125,336

(22) Filed: May 10, 2005

Related U.S. Application Data
(60) Provisional application No. 60/569,921, filed on May 10, 2004.
(60) Provisional application No. 60/583,670, filed on Jun. 28, 2004.

(51) Int. Cl. A61F 2/00

(52) U.S. Cl. 424/423

(57) ABSTRACT

The present invention is directed to a biostructure comprising an osteoconductive member and an osteoinductive material. The osteoinductive material may be located within a cavity in the osteoconductive material. In one aspect of the invention the osteoinductive material is demineralized bone matrix and the osteoconductive member comprises tricalcium phosphate.

This application claims priority under 35 U.S.C. § 119(e) to U.S. Provisional Application 60/569,921, filed on May 10, 2004, and U.S. Provisional Application 60/583,670, filed on Jun. 28, 2004, both of which are incorporated by reference in their entirety.

### BACKGROUND OF THE INVENTION

1. Field of the Invention

The invention pertains to implants for the healing and regeneration of bone and more particularly to an osteoconductive matrix having selective deposits of demineralized bone in channels, passageways, cavities and lumens of the matrix.

2. Description of the Related Art

Implants to encourage the regeneration and healing of bone have come into increasing use. Among the materials used have been autograft (the patient's own bone), allograft (bone from deceased human donors), and synthetic materials such as members of the calcium phosphate family.

Synthetic ceramic materials have been shown to be osteoconductive, i.e., able to conduct the ingrowth of natural bone when placed against adjacent natural bone. This ability is a function primarily of the chemistry and also of the geometry (pore size, etc.) in which the materials are manufactured. Some synthetic ceramic materials are resorbable, meaning that they can eventually disappear through normal biochemical processes and be replaced by natural bone. Implantable ceramic structures have been made for this purpose by three-dimensional printing, by molding and by other methods.

Another useful material has been demineralized bone matrix, which was shown by Urist in 1965 to have properties of stimulating the differentiation of bone progenitor cells into actual bone cells. This property has been termed osteoinductivity. In order to be osteoinductive, demineralized bone matrix has to exist in the form of particles greater than a certain minimum size, typically 100 micrometers. Demineralized bone matrix has been made into a major component of putty, sheet, and other forms which have been flexible, because demineralized bone matrix basically is a soft or spongy material, especially when it is wet. Putty has been suitable to be applied directly to bones during surgical repair. A limited number of solid implant biostructures have been made by molding demineralized bone matrix with a binder. In regard to osteoinductive additives which are not discrete particles, there are also other substances which are known to be osteoinductive, such as bone morphogenetic protein, transforming growth factor beta, etc.

A combination of osteoinductivity and osteoconductivity is disclosed in U.S. Pat. No. 6,695,882. In that patent, which pertains to spinal fusion surgery, it is described that a chamber in a dowel derived from natural bone allograft may be packed with an osteogenic material composition which is described as “including autograft, allograft, xenograft, demineralized bone, synthetic and natural bone graft substitutes, such as bioceramics and polymers, and osteoinductive factors.” However, the fact that this material is described as being packed into a chamber indicates that it does not have definite form.

Elsewhere, the combination of osteoinductivity and osteoconductivity in structures has been accomplished in the sense of soaking a porous osteoconductive structure with an osteoinductive liquid, which occupies pores in the structure. The liquid has contained osteoinductive substances such as bone morphogenetic proteins. However, this approach has only been applicable to osteoinductive substances which are liquids.

In Induction of Bone by a Demineralized Bone Matrix Gel: A Study in a Rat Femoral Defect Model, by John E. Feighan, Dwight Davy, Annamarie Prewett, and Sharon Stevenson, Journal of Orthopaedic Research 13, No. 6, 1995, pp. 881-891); and in A Coralline Hydroxyapatite and Demineralized Bone Matrix Gel Composite for Bone Grafting, by Christopher J. Damien, J. Russell Parsons, Annamarie B. Presett, Frank Huismans, Michael Vanazio and Edwin C. Shors, excerpted from the Fourth World Biomaterials Congress, Apr. 24-28, 1992, Berlin, there is disclosed a porous matrix of a calcium phosphate material whose pores have contained a gel of particles of demineralized bone matrix in a glycerol carrier. The process described in those publications has required that the pores be sufficiently large and the DBM particles be sufficiently small so that the DBM particles can enter the pores. This has involved an inherent conflict or mismatch of dimensional scales. Osteoinductivity of DBM particles generally requires a particle size of at least 100 micrometers, and ability to place particles in pores such as by flowing gel into pores would require that the pores be larger than the DBM particles by some factor. All of this would tend to require pore sizes of at least several hundred micrometers. However, for cell and tissue ingrowth into the pores, it would be desirable for the pore size to be approximately 100 micrometers or smaller. With a conventional biostructure which is of uniform architecture, it has not been possible to satisfy both of these requirements simultaneously.

This conflict in terms of desired pore size has worked against the optimum use of demineralized bone matrix, which is an excellent osteoinductive material, in rigid osteoconductive structures.

Accordingly, it would be desirable to provide a biostructure having a definite structure which is both osteoconductive and osteoinductive, by having a structure which is osteoconductive and which contains particles of demineralized bone matrix as the osteoinductive material. It would be desirable for the DBM particles to be contained in internal features which are sufficiently large to contain the DBM particles, while at the same time providing pores which are smaller than the particles of DBM, which are suitable for the ingrowth of cells and tissue. It would be desirable for the structure to comprise members of the calcium phosphate family such as tricalcium phosphate. It would be desirable for particles of demineralized bone matrix, besides occupying appropriate places, be affixed in those places such that the particles of DBM do not readily move away. It would be desirable for such a biostructure to be able to be manufactured by three-dimensional printing.

### BRIEF SUMMARY OF THE INVENTION

The biostructure includes a porous matrix, which may be osteoconductive and may comprise a ceramic such as tricalcium phosphate. In some embodiments, the matrix may comprise polymer or may comprise both ceramic and polymer. The matrix also may comprise one or more channels, recesses or internal region(s), whose size is larger than the size of pores, with the channels, recesses or internal region(s) being suitably dimensioned so as to contain osteoinductive material. The biostructure also may comprise particles of osteoinductive material such as demineralized bone matrix, which may exist in the form of particles greater than a certain minimum size. The particles of demineralized bone matrix may be contained in the interior of the biostructure, or may be attached to the exterior of the biostructure, or both. The biostructure may further comprise another material which holds the osteoinductive particles in place. The biostructure can have a shape suitable for use as any of a variety of bone replacements and can be suitable to be carved at the point of use and suitable to wick bodily fluids. The invention also includes methods of manufacturing such a biostructure. The particles of demineralized bone matrix may be added at a stage later than the manufacturing of the matrix. The biostructure may assembled from more than one piece.

In one embodiment, the invention relates to a biostructure comprising an osteoconductive member defining at least a first macroscopic feature; and a material comprising osteoinductive material within the first macroscopic feature. The first macroscopic feature may be in the form of an interior void or cavity, an external void or cavity, a through-channel, a dead-ended channel, a recess, or an indentation. The osteoconductive member may comprise pores whereby the osteoinductive material is accessible to bodily fluids from outside of the biostructure through the pores of the osteoconductive member. In another embodiment, the invention relates to a biostructure comprising: an osteoconductive member having a first dimension; and a coating of material comprising osteoinductive particles on at least a portion of the surface of the osteoconductive member, wherein the coating has a second dimension that is less than the first dimension. In another embodiment, the invention relates to a method of manufacturing a biostructure, the method comprising: providing an osteoconductive member; and depositing a material comprising osteoinductive particles in or on the osteoconductive member.

### BRIEF DESCRIPTION OF THE SEVERAL VIEWS OF THE DRAWINGS

Embodiments of the invention are illustrated in the Figures herein.

FIG. 1a shows a rectangular prismatic biostructure with a centrally-located through-channel which contain particles of DBM. FIG. 1b is a cross-section of FIG. 1a. FIG. 1c shows a similar biostructure with two dead-ended channels which contain DBM.

FIGS. 2a, 2b, and 2c illustrate similar macro-channel features in biostructures which are of overall cylindrical shape.

FIG. 3 shows biostructures which contain a macroscopic interior void which is connected to the exterior of the biostructure by a macrochannel.

FIG. 4 shows components of a biostructure which can contain a macroscopic interior void which does not need to be connected to the exterior of biostructure by a macrochannel. This shows a biostructure which is assembled from sub-components capable of being joined, formed together, etc.

FIGS. 5, 6 and 7 show still further designs of biostructures which can contain macroscopic internal cavities and which involve closure by a closure sub-component.

FIG. 8 illustrates biostructures with a central region which can contain DBM and further containing either through-channels or dead-end channels from other directions.

FIG. 9 illustrates the placement of particles of DBM on exterior surfaces of a biostructure.

### DETAILED DESCRIPTION OF THE INVENTION

The invention includes a biostructure having an overall shape. The biostructure may, first of all, comprise a matrix which is porous. The pores may be characterized by pore sizes which may be in the range of approximately 1 micrometer to approximately 1000 micrometers. In certain embodiments, the pore size distribution has a peak between 50 and 100 micrometers. In one embodiment the matrix may comprise particles which are partially joined directly to each other but still leave some space between themselves in the form of pores. In another embodiment the matrix may comprise particles which are joined to each other by another substance(s). In any embodiment the matrix may be osteoconductive, such as by virtue of the geometry and/or composition of the matrix.

The matrix may further include macroscopic channels which are suitable to be occupied by particles of DBM. The macroscopic channels may have cross-sectional dimensions which, first of all, are greater than approximately three times the average pore diameter, so that the macroscopic channel is distinguishable as being different from a pore. Edges of the matrix may define boundaries of the macroscopic channels. Further, the macroscopic channels may have dimensions which are greater than the dimensions of usefully sized particles of demineralized bone matrix, as described elsewhere herein. The channels may include channels open at both ends, blind channels, surface features resembling tire treads, straight channels, channels with curves or changes of direction, constant-cross-section channels, tapered channels, and intersecting channels. Cross-sectional dimensions of such channels may, for example, be greater than approximately 100 micrometers or in some embodiments greater than approximately 300 micrometers Channels may or may not traverse completely through the matrix, i.e., channels may be either open-ended (through-channels) or closed-ended (dead-ended). Dead-ended channels may be of any depth (length) relative to their cross-sectional dimension, i.e., they may be deep, or they may be shallow, resembling surface indentations. Also the channels may lie along various different planes or have different directions, in any relative combination and orientation. The cross-sectional shape of the channels may be cylindrical, rectangular, or other shape.

The biostructure may further contain macroscopic internal voids which are suitable to be occupied by particles of DBM. A macroscopic internal void may be an internal region not occupied by matrix, which has a cross-sectional dimension of at least 200 micrometers and in some embodiments at least 400 micrometers. The macroscopic internal voids may have cross-sectional dimensions which, first of all, are greater than approximately three times the average pore diameter, so that the macroscopic internal void is distinguishable as being different from a pore. In some embodiments, macroscopic internal voids may be connected by at least one channel to the exterior, but in other embodiments it is not necessary.

In some embodiments, macroscopic internal voids may have access or connection to the exterior surface of the biostructure. In such instance, macroscopic internal void may have a cross-sectional dimension which is larger than the cross-sectional dimension of the associated macroscopic channel. In other embodiments, which may be manufactured by methods described elsewhere herein, it is not necessary for a macroscopic internal void to be connected to the exterior.

The biostructure can also include an osteoinductive material. The osteoinductive material may exist in the form of particles of a solid, which may be particles of demineralized bone matrix (DBM). The particles of DBM may have overall dimensions which are greater than what is believed to be a minimum dimension for DBM to have osteoinductive properties without causing any appreciable inflammatory response in the body. For example, the particles of DBM may have dimensions of at least approximately 100 micrometers. The particles of DBM may have overall dimensions which are in the range of approximately 100 micrometers to 800 micrometers, as is typical in the demineralized bone matrix art.

Particles of DBM can be located within macroscopic channels, or may be located within macroscopic internal voids, or both. Any such location of DBM may be helpful for keeping the particles of DBM located physically within the biostructure and also may be helpful for providing a sustained action of the DBM in stimulating the growth of bone. There may be a time delay associated with the entry of bodily fluids into the interior of the biostructure where the DBM is located, and also with the exit of bodily fluids from the DBM-containing region. Particles of DBM which are within the biostructure, either inside macroscopic channels or inside macroscopic internal voids, either may be loosely contained within those features or may be attached to the matrix by an attachment material.

The biostructure may also have particles of DBM attached to the exterior of the matrix. Such location of DBM particles may be helpful in providing a more immediate action of DBM in stimulating the growth of bone, because external DBM would be readily exposed to bodily fluids, and substances leaving the DBM could readily contact adjacent tissue. Particles of DBM which are attached to the exterior may be attached by an attachment substance.

The biostructure may contain any or all of the above placements of particles of DBM in any combination, thereby providing a combination of immediate release and longer-duration release of substances derived from DBM.

As mentioned, in the finished biostructure, particles such as of Demineralized Bone Matrix may be contacted by, or may be either fully or partially surrounded by, a further substance which may be designated an attachment substance. Such attachment substance may attach particles of DBM to the matrix itself or to other particles of DBM which may or may not be attached to the matrix. It is possible that the attachment substance can be a dry solid. Such dried condition may be a robust condition for shipping and handling of a composite biostructure. Alternatively, it is also possible that the attachment material, in which the DBM exists, may be present in moist or deformable form such as in the form of a paste or gel or viscous liquid.

In addition to DBM, other osteoinductive materials are contemplated, some of which are both osteoconductive and osteoinductive. For instance, fully or partially demineralized bone matrix materials may be used. In addition bone chips such as cancellous chips may be used.

It is possible that in a biostructure containing macroscopic channels and/or macroscopic internal voids, some of those features may contain particles of DBM while other such features do not. The channels which do not contain particles of DBM may be provided for the purpose of conducting the ingrowth of tissue or providing place for blood vessels to grow. Such features are believed to be helpful for promoting ingrowth and integration. Macroscopic channels which do not contain particles of DBM may have cross-sectional dimensions which are smaller than the dimensions of particles of DBM, or are smaller than the cross-sectional dimensions of macroscopic channels which do contain particles of DBM.

The biostructure may have more than one sub-component making up the matrix, and the sub-components may physically either fit together or interlock with each other, or the sub-components may be attached to each other. For example, it is possible that a first sub-component may be a shape made of porous material and having a cavity and an aperture, and a closure sub-component may extend to close the aperture. The closure sub-component may be mechanically interlocking with the first sub-component, or may be glued or fused to the first sub-component. For example, some of the closure sub-component may occupy some pores of the first sub-component as a way of attaching itself to the first sub-component.

It is further possible that space which is not occupied by any of the described materials (structure such as tricalcium phosphate, particles demineralized bone matrix, attachment substance such as gelatin) could be occupied by still other materials. More specifically, such substance could be an Active Pharmaceutical Ingredient. Examples of categories of Active Pharmaceutical Ingredients which could be included are angiogenic factor (to promote the growth of blood vessels), antibiotics (to counteract infection) and anesthetics (for pain relief). A still further category of substances which could be added, to provide added osteoinductivity, is an Active Pharmaceutical Ingredient which stimulates the formation of bone, such as by stimulating the formation of bone morphogenetic protein. Examples of such substances include the family of HMG-CoA reductase inhibitors, more specifically including the statin family such as lovastatin, simvastatin, pravastatin, fluvastatin, atorvastatin, cerivastatin, mevastatin, and others, and pharmaceutically acceptable salts esters and lactones thereof. As far as lovastatin, the substance may be either acid form or the lactone form or a combination of both.

As examples of other materials which may be included in the biostructure, the osteoconductive matrix may comprise one or more members of the calcium phosphate family. Tricalcium phosphate, which is resorbable, may be used. Tricalcium phosphate exists in the crystal forms beta and alpha, of which beta is believed to have a more desirable (slower) resorption rate. Hydroxyapatite may be used, as may still other members of the calcium phosphate family. Other ceramics may be used. In a ceramic matrix, particles of ceramic may be joined directly to other particles of ceramic, such as by necks made of ceramic material which may be the same material as the particles themselves.

Other biocompatible materials may also be used. Polymers, either resorbable or nonresorbable or a combination thereof, may be used. The matrix could be made of a combination of polymer and osteoconductive material. The osteoconductive material could exist in the form of particles of ceramic, and the matrix could be an overall matrix of polymer, containing pores, which also holds particles of the ostoconductive substance. The following polymers are suitable for making an osteoconductive matrix: polylactones, polyamines, polymers and copolymers of trimethylene carbonate with any other monomer, vinyl polymers, acrylic acid copolymers, polyethylene glycols, polyethylenes, Polylactides; Polyglycolides; Epsilon-caprolactone; Polylacatones; Polydioxanones; other Poly(alpha-hydroxy acids); Polyhydroxyalkonates; Polyhydroxybutyrates; Polyhydroxyvalerates; Polycarbonates; Polyacetals; Polyorthoesters; Polyamino acids; Polyphosphoesters; Polyesteramides; Polyfumerates; Polyanhydrides; Polycyanoacrylates; Poloxamers; Polysaccharides; Polyurethanes; Polyesters; Polyphosphazenes; Polyacetals; Polyalkanoates; Polyurethanes; Poly(lactic acid) (PLA); Poly(L-lactic acid) (PLLA); Poly (DL-lactic acid); Poly-DL-lactide-co-glycolide (PDLGA); Poly(L-lactide-co-glycolide) (PLLGA); Polycaprolactone (PCL); Poly-epsilon-caprolactone; Polycarbonates; Polyglyconates; Polyanhydrides; PLLA-co-GA; PLLA-co-GA 82:18; Poly-DL-lactic acid (PDLLA); PLLA-co-DLLA; PLLA-co-DLLA 50:50; PGA-co-TMC (Maxon B); Polyglycolic acid (PGA); Poly-p-dioxanone (PDS); PDLLA-co-GA; PDLLA-co-GA (85:15); aliphatic polyester elastomeric copolymer; epsilon-caprolactone and glycolide in a mole ratio of from about 35:65 to about 65:35; epsilon-caprolactone and glycolide in a mole ratio of from about 45:55 to about 35:65; epsilon-caprolactone and lactide selected from the group consisting of L-lactide, D-lactide and lactic acid copolymers in a mole ratio of epsilon-caprolactone to lactide of from about 35:65 to about 65:35; Poly(L-lactide and caprolactone in a ratio of about 70:30); poly (DL-lactide and caprolactone in a ratio of about 85:15); poly(DL-lactide and caprolactone and glycolic acid in a ratio of about 80:10:10); poly(DL-lacticde and caprolactone in a ratio of about 75:25); poly(L-lactide and glycolic acid in a ratio of about 85:15); poly(L-lactide and trimethylene carbonate in a ratio of about 70:30); poly(L-lactide and glycolic acid in a ratio of about 75:25); Gelatin; Collagen; Elastin; Alginate; Chitin; Hyaluronic acid; Aliphatic polyesters; Poly(amino acids); Copoly(ether-esters); Polyalkylene oxalates; Polyamides; Poly(iminocarbonates); Polyoxaesters; Polyamidoesters; Polyoxaesters containing amine groups; and Poly(anhydrides). The polymer can also be copolymer or terpolymer. It can be a blend of two or more individual substances mixed together.

Some embodiments comprise a closure sub-component in addition to a first sub-component. The closure component or additional sub-component may be made of whatever the first sub-component is made of, such as a porous ceramic. Alternatively, the closure sub-component could be made of gelatin, such as porcine gelatin, which may be dried. The gelatin could additionally contain particles of osteoconductive material such as a calcium phosphate.

It is possible that the attachment material can be a dry solid. This dried condition may be a robust condition for shipping and handling of a composite biostructure. An example of such a attachment material is dehydrated gelatin. Alternatively, it is also possible that the attachment material, in which the DBM exists, may be present in moist form such as in the form of a paste or gel or viscous liquid. The attachment material may include particles of osteoconductive material such as a calcium phosphate.

The biostructure can have a shape suitable for use as any of a variety of bone replacements and can be suitable to be carved at the point of use. The porosity and the physical properties of ceramics such as tricalcium phosphate makes the material easily carvable for dimensional adjustment during surgery. Porosity as described herein further causes the material to be able to wick and retain blood, marrow and other aqueous bodily fluids.

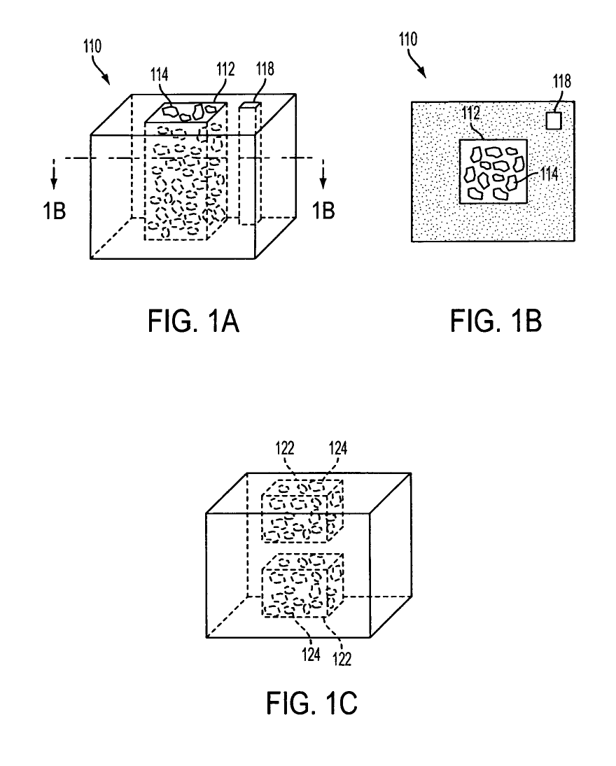

The biostructure could be supplied in the form of an aggregate of a number of such biostructures, which may be identical with each other or may differ such as in dimensions or shape. The aggregate may be suitable to be poured or packed into a void in a bone, or mixed with still other substances or biostructures and placed in a void in a bone. The individual biostructures making up the aggregate could, for example, be of cruciform prismatic shape. Such shapes and aggregates are described in commonly assigned co-pending U.S. patent application Ser. No. 10/837,541 (docket number 44928.210), which is hereby incorporated by reference. Embodiments of the invention are further described in the Figures. FIG. 1a and 1b shows a rectangular prismatic matrix 110, with FIG. 1b being a cross-section of FIG. 1a. Centrally located within matrix 110 is a through-channel 112. Contained inside the through-channel 112 are particles 114 of demineralized bone matrix. An attachment material (not shown) may also be present, joining the particles of DBM to the matrix and/or each other. Exterior side lengths of the matrix may be, for example, 0.5 cm to 2 cm, and may be unequal if desired. Also shown is another smaller through-channel 118 which does not contain any particles of DBM. Channel 112 which contains particles of DBM may have dimensions such as approximately 0.5×0.5 mm or larger. Matrix 110 is also shown as containing channel 118 which does not contain DBM. Channels such as channel 118 may have dimensions smaller than the channel 112 which contains particles of DBM. For example, the non-DBM-containing channel 118 may have cross-sectional dimensions which are smaller than the dimension of some of the particles of DBM. Such channel cross-section dimensions could be as small as approximately 0.1 mm×0.1 mm (100 micrometers×100 micrometers). FIG. 1c shows a similar matrix 120 which has a dead-end channel 122 in the top face and a similar dead-end channel 122 in the bottom face. Both of these channels 122 contain particles of DBM 124. Again, there may be an attachment material (not shown) which may physically connect DBM particles 124 to the wall of the channel 122 or to other DBM particles or both. Although the rectangular block shaped biostructures illustrated in FIG. 1 appear roughly cubical, they could be of any proportion.

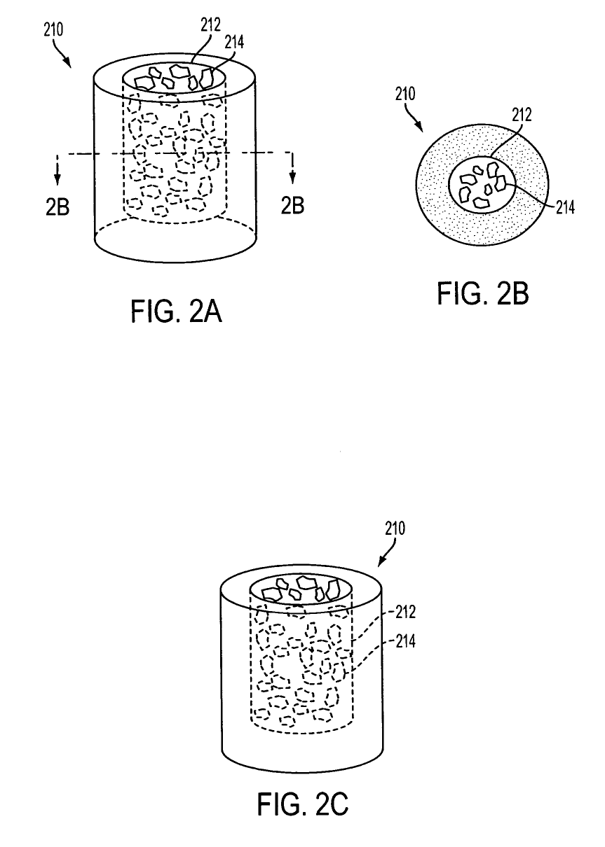

FIG. 2 illustrates features which are generally similar to those illustrated in FIG. 1, but illustrates them for biostructures which are of overall cylindrical shape. In FIG. 2a and in FIG. 2b (which is a cross-section of FIG. 2a), the biostrcture 210 is cylindrical shaped, and the channel 212 is a through-channel which also has a cylindrical cross-section. In FIG. 2c, the channels are dead-end channels. In FIG. 2c the dead-end channels are shown non-coaxial with the cylinder, but in general they could be of any orientation.

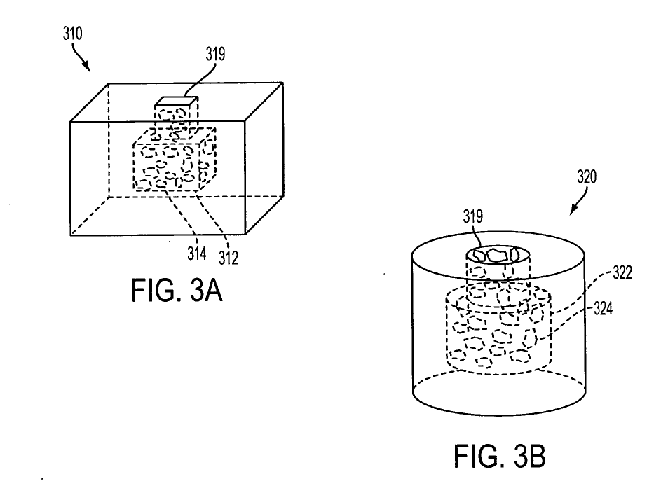

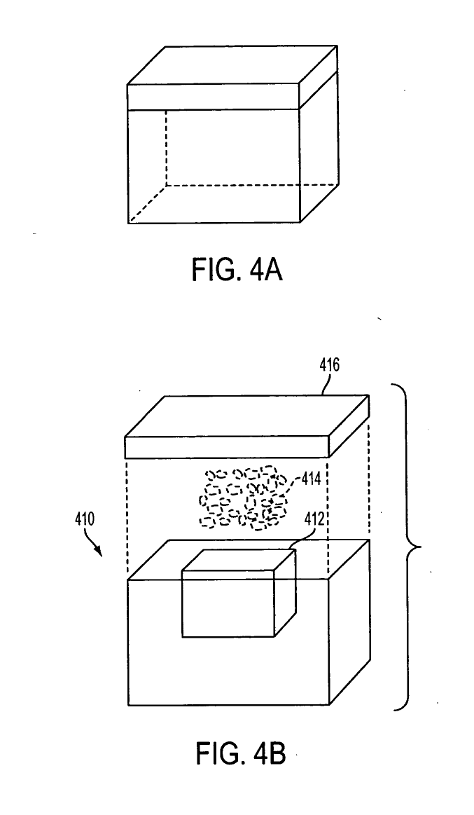

FIG. 3 shows some biostructures 310 which contain a macroscopic interior void 312 which is connected to the exterior of the biostructure by a macroscopic channel 319. If a macroscopic interior void is connected to the exterior by a macroscopic channel having channel cross-sectional dimensions, the dimensions of the macroscopic interior void may be larger in at least one dimension than the cross-sectional dimensions of the macroscopic channel. FIG. 3 shows particles of DBM 314 are contained inside the macroscopic interior void 312 and also inside the macroscopic channel 319. The overall biostructure may be cylindrical or rectangular in shape and may have an overall volume of approximately 0.5 to 5.0 cm3. The matrix may have multiple macroscopic internal voids and/or macroscopic channels, and either all or some of the voids or channels may contain particles of DBM. Macroscopic internal voids which contain particles of DBM may have dimensions of at least approximately 0.5 mm×0.5×0.5 mm. It is not actually necessary that a macroscopic interior void be connected to the exterior by a macroscopic channel (although this choice would impact the manufacturing process). In this case, the macroscopic interior void is formed in an inner wall of the osteoconductive member. As an alternative, the osteoconductive matrix may comprise two or more pieces which fit together to form a desired biostructure. A biostructure which contains multiple sub-components is shown in FIG. 4. The two or more sub-components may cooperate so as to provide interior empty space suitable to be occupied by the osteoinductive material such as particles of DBM. In a design having multiple sub-components, the macroscopic interior voids or macroscopic channels could have access to the exterior as it did in the previous design, but it is not required that such access exist. A two-sub-component design without such access is shown in FIG. 4. The individual sub-components could be suitable to connect to each other by adhesive, by mechanical interlock, or by other means. FIG. 4 illustrates a body 410 which contains a recess 412 and which also is suitable to be used with a lid 416. FIG. 4 also shows this biostructure assembled with DBM particles 414 located in the recess 412.

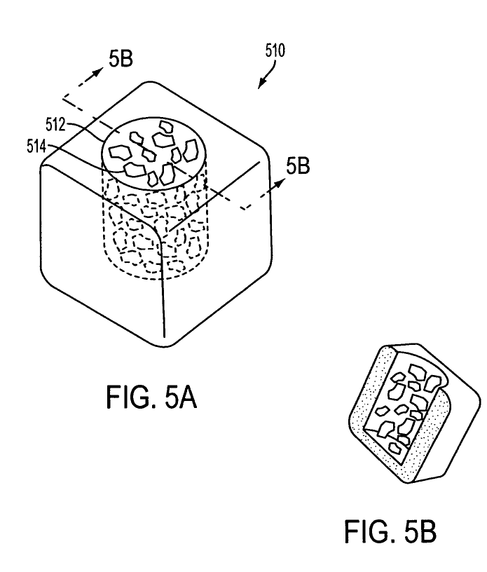

FIG. 5a shows another design of a cavity-containing matrix which is suitable to be closed by a cap. The matrix in FIG. 5 is a simple rectangular prism 510 with a single cylindrical dead-ended cavity 512 through one face. FIG. 5b is a cross-section of FIG. 5a. There can also be particles 514 of DBM in cavity 512. The DBM particles would be in the cavity, possibly along with dried gelatin or other attachment material holding the DBM particles in place. In FIG. 5 there is not any special feature to receive the cap.

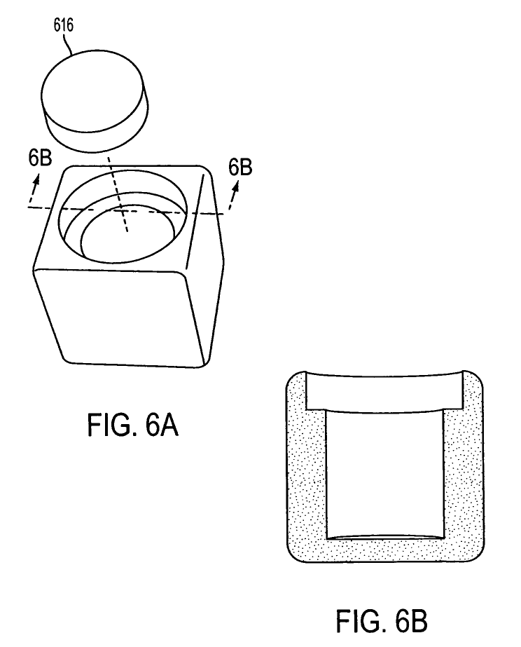

FIG. 6 is similar to FIG. 5 but further illustrates a feature suitable to receive a cap. There is shown a recessed lip, which is shown as being round and of a larger cross-section than the cylindrical dead-ended cavity itself. The lip is to shaped suitably accommodate a cap or formable closure. The cap or formable closure could contain or be made of gelatin. As in previous illustrations, the DBM particles 614 in the cavity 612 could be loose dry DBM particles or they could exist together with attachment material such as gelatin which helps hold them in place. The attachment material could be either dried or could still be wet or deformable. FIG. 6a is a view of the biostructure and FIG. 6b is a cross-section of FIG. 6a.

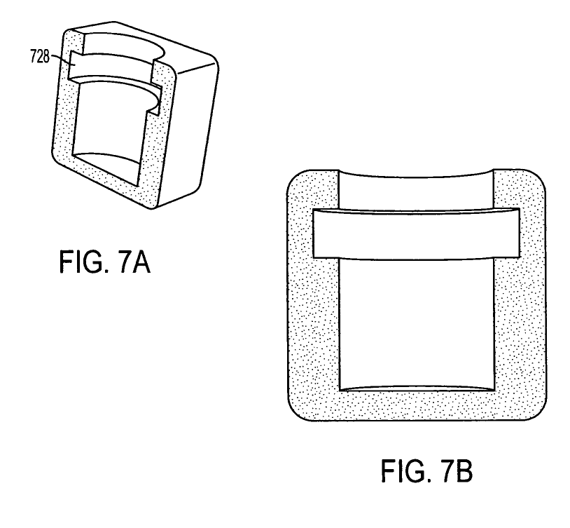

FIGS. 7a and 7b (with 7b being a cross-section of 7a) show a still slightly different design of the lip, in which the lip would physically trap the cap or closure component. In this figure, the lip is really a groove which can trap the cap in place by virtue of the geometry of the groove.

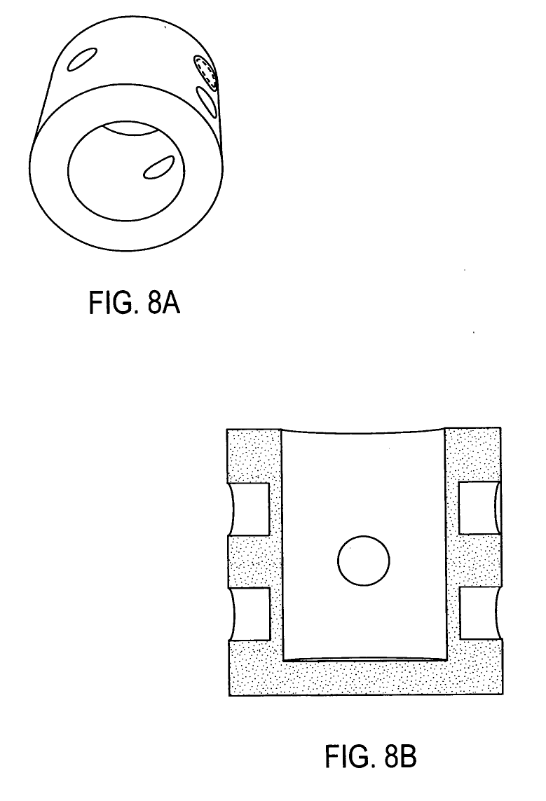

FIG. 8 shows a hollow cylindrical osteoconductive matrix which also has some holes through the wall or dimples partially into the wall. As in previous illustrations, DBM could occupy any of the empty spaces where it is desired to have DBM. There could be attachment material such as dried gelatin among the DBM particles, caps as previously described, etc. Alternatively, the DBM particles could be dry loose particles without attachment material among them. The adjacent illustration in this same Figure shows the same as the previous illustration except that osteoconductive matrix is closed at one end. This Figure further illustrates that external surfaces of the biostructure could contain concave features such as dead-ended macroscopic channels which are not very deep, which are suitable to contain particles of DBM.

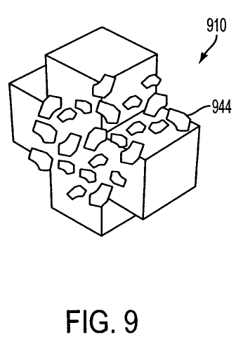

FIG. 9 shows an osteoconductive matrix which contains particles of DBM on external surfaces of the matrix. This can be done even if the external surfaces of the matrix do not contain any concave features. The particles of DBM could be attached onto the surface of the osteoconductive matrix by an attachment substance, such as by dried gelatin. Any of the shapes described herein, or any other shapes, could have DBM particles on their exteriors. Such attachment of DBM particles onto external surfaces could be done onto any of the biostructures which also contains DBM internally, such as have previously been described.

Method of Manufacturing

The invention also includes methods of manufacturing such a biostructure.

The method may include three dimensionally printing the matrix.

The method of manufacturing the matrix may include the use of a decomposable porogen such as lactose.

The method of manufacturing may include chemical reaction from precursors. For example, hydroxyapatite, which is Ca₁₀(PO₄)₆(OH)₂, plus dicalcium phosphate, which is Ca H PO₄, upon being heated, yields tricalcium phosphate. However it is not necessary to involve a chemical reaction; it is also possible to simply spread ceramic powder of the desired final composition and perform three-dimensional printing on that powder.

Besides three-dimensional printing, other methods of forming the matrix are also possible. For example, it is also possible to form the matrix by molding or by material removal methods (e.g., drilling holes) or by a combination of any of the methods discussed herein.

After the manufacturing of a preform containing ceramic, the preform may be heated to cause the ceramic particles to partially join directly to each other, i.e., sintering. The heating may also cause decomposition of the particles of decomposable porogen, and may cause chemical reaction between reactants if reactants are provided.

Alternatively, the matrix may be manufactured by forming a matrix of organic-solvent-soluble material such as polymer. This can involve causing particles of an organic-solvent-soluble substance such as a polymer to join to each other. The matrix of organic-solvent-soluble material may contain particles of ceramic such as one or more members of the calcium phosphate family. Formation of such an article can also involve three dimensional printing, such as by dispensing organic solvent from the printhead.

After the manufacturing of the matrix, the method of the present invention may further include introducing particles of DBM into or onto appropriate places in the matrix. It is possible that loose particles of DBM may simply be physically placed in desired locations. For example, if the design contains multiple sub-components, such loose particles of DBM may be retained in place by assembly or closure. Alternatively, a paste, viscous liquid, gel etc. comprising a carrier together with the osteoinductive material such as particles of demineralized bone matrix may be placed in desired places. The eventual attachment material may be the carrier or a dried form of the carrier, or could be a different material. In particular, the particles of DBM may be contained in a carrier which may be gelatin. Gelatin has known biocompatibility, resorbability and similar advantages. The gelatin may be porcine gelatin or gelatin from some other source. The introducing could be done by injecting with a syringe, for example. This step may be followed by dehydrating the paste, viscous liquid, gel etc., so as to leave a relatively solid, dry substance in contact with the particles of DBM. The dehydrating can be performed by lyophilizing. Lyophilization (freeze-drying) is a known process for use in preparing demineralized bone matrix. However, if desired, the invention can be practiced without a drying step. If the biostructure comprises a matrix which is made in more than one sub-component, the sub-components may be joined together at approximately this point in the manufacturing process.

The carrier which has been described so far (gelatin) has been water-based. Water-based carriers are typical of bone putties that are placed directly inside the human body in the form of putty. However, it can be noted that it is possible for the attachment material to be chemically based on a solvent or liquid other than water, such as a solvent or liquid which might not be appropriate for exposure to the body of a patient. For example, the carrier could include a solvent such as alcohol or chloroform which would probably not be desirable for exposure to the body of a patient. This is possible because after the introduction of the paste, viscous liquid or gel into the matrix, there are subsequent manufacturing steps and opportunities to remove any objectionable substances such as by evaporation.

Another alternative manufacturing process could involve carrying the osteoinductive particles into place using a first attachment material, removing that first attachment material such as by dissolving it and rinsing it away, and then introducing a second substance suitable to remain in place as an attachment material which may hold the osteoinductive particles in place. This second substance can be dried such as by lyophilizing, if desired. The first attachment material could be a hydrocarbon-based grease or fat, for example. Such substances are soluble in chloroform and other solvents for possible removal. Demineralized bone matrix is known to be undamaged by chloroform, because chloroform is used in its manufacture. The second substance, which may be an attachment material suitable to hold the DBM particles in place in the finished product, could be gelatin or other suitable substance which is suitable to remain in the finished product and be implanted into the human body. Yet another method could comprise simply placing particles of demineralized bone matrix in appropriate places and then introducing gelatin or attachment material to help hold the particles of DBM in place. This could be followed by a drying step.

Manufacture of articles of the present invention can involve assembling the articles from sub-components. Articles which are made at least in part from particles of polymer or similar organic-solvent-soluble material can also be manufactured by yet another method. If the matrix contains organic-solvent-soluble substance such as polymer, it is possible that two or more sub-components could be made individually, and then particles of DBM could be placed in what would become the interior of the assembled biostructure, and then the two or more sub-components could be joined to each other after the DBM is in place, thereby enclosing the DBM. For example, chloroform is a solvent for many polymers. Also, it is known that chloroform does not damage DBM, because typically chloroform is already used in the manufacture of DBM. When two or more sub-components of the matrix are touching each other or interlocking as desired in the final configuration, it is possible to use exposure to organic solvent such as chloroform to cause the sub-components to fuse with each other or join each other, by exposing the assembled sub-components to the organic solvent and then removing the organic solvent. For example, the assembled sub-components can be exposed to liquid organic solvent or vapor of the organic solvent or both, either locally or throughout. The organic solvent could, for example, be chloroform. Local application of liquid organic solvent can take the form of applying liquid to the joint region in much the same way as liquid glue would be applied in repairing a broken object. The liquid could further contain, dissolved in it, a polymer or other substance which would act as an adhesive. If the dissolved substance is a polymer, it could be either the same polymer present in the structure or a different polymer. In fact, if this is done, the structure or some of its sub-components do not even have to contain polymer; it might be sufficient for polymer which is contained in solution in liquid organic solvent to adhere the pieces together. At least some of the organic solvent can be removed through evaporation. If further removal is needed, it can be accomplished by exposure to carbon dioxide, or other suitable substance in a supercritical or critical state, or to a liquid form of carbon dioxide (pressurized to an appropriate pressure) or other suitable substance.

Biotructures such as the biostructure in FIG. 7 could be made by pouring gelatin into place to take the shape of the cap region including the groove. In order to help make the gelatin flowable or formable, the gelatin could be warmed such as to above body temperature. After the gelatin is poured into place, the gelatin could harden either by cooling down or by drying or both. In a related detail, the cap or formable closure could be pre-made in an approximate size and shape, and could be softened and put into place and allowed to harden. The cap or formable closure does not have to be pure gelatin but could also contain, for example, particles of tricalcium phosphate or other osteconductive material (or could even contain DBM particles as well).

For biostructures which contain DBM particles attached to their exterior, the biostructure could be made by manufacturing the osteoconductive matrix, and then applying to the external surfaces of the osteoconductive matrix a paste or gel containing the DBM particles in a carrier substance. This could be done by applying a DBM+gelatin gel onto the external surface, or by applying gelatin or other gel to the external surface and then exposing the gelatin to DBM powder, such as by rolling the article around in an aggregate of the DBM particles so that DBM particles stick and become attached. The carrier substance could be allowed to dry out. For articles which contain DBM particles attached to their exterior, the DBM particles could be applied as just described above. Drying could be at room or warm conditions or could be freeze-drying.

After all of the steps so far described, it is still possible to introduce still other substances into the article, such as by soaking. Such substances could be any Active Pharmaceutical Ingredient or other bioactive substance, as described elsewhere herein.

Sterilization may be accomplished by any of several means and sequences in relation to the overall manufacturing process. The overall manufacturing process may include terminal sterilization, which would be sterilization after completion of all other manufacturing steps including the placement of the osteoinductive material. Such a terminal sterilization method may include irradiation. The irradiation may be by electron beam, which is known to induce less damage to biological substances than gamma irradiation, or the irradiation may be by a sufficiently low dose of gamma radiation.

Another possible manufacturing sequence is that the osteoconductive matrix may be manufactured by any suitable means and may be sterilized by any suitable means, and then all subsequent processing steps, such as introducing the osteoinductive material, may be performed in aseptic conditions. An advantage of this sequence is that the osteoconductive matrix, such as ceramic, may be sterilized by aggressive sterilization methods which would not be permissible as terminal sterilization processes if the osteoinductive material were already present.

Other embodiments and uses of the invention will be apparent to those skilled in the art from consideration of the specification and practice of the invention disclosed herein. All references cited herein, including all U.S. and foreign patents and patent applications, are specifically and entirely hereby incorporated herein by reference. It is intended that the specification and examples be considered exemplary only, with the true scope and spirit of the invention indicated by the following claims.

1. A biostructure comprising:
an osteoconductive member defining at least a first macroscopic feature; and
a material comprising osteoinductive material within the first macroscopic feature.

2. The biostructure of claim 1, wherein the first macroscopic feature is in the form of an interior void or cavity, an external void or cavity, a through-channel, a dead-ended channel, a recess, or an indentation.

3. The biostructure of claim 1, wherein the first macroscopic feature is defined by an outer surface of the osteoconductive member.

4. The biostructure of claim 1, wherein an inner surface of the osteoconductive member defines the first macroscopic feature.

5. The biostructure of claim 1, wherein the osteoconductive member comprises an outer surface and a channel defining an inner surface of the osteoconductive member.

6. The biostructure of claim 5, wherein the inner surface of the osteoconductive member comprises an inner wall, and the first macroscopic feature is formed in the inner wall of the osteoconductive member.

7. The biostructure of claim 5, wherein the outer surface of the osteoconductive member comprises an outer wall, and the first macroscopic feature is formed in the outer wall of the osteoconductive member.

8. The biostructure of claim 5, wherein the inner surface of the osteoconductive member comprises an inner wall and the first macroscopic feature is formed in the inner wall of the osteoconductive member, the outer surface of the osteoconductive member comprises an outer wall and a second macroscopic feature is formed in the outer wall of the osteoconductive member.

9. The biostructure of claim 1, wherein the osteoconductive member comprises pores and the osteoinductive material is accessible to bodily fluids from outside of the biostructure through the pores of the osteoconductive member.

10. The biostructure of claim 1, further comprising a cap or formable closure which encloses the osteoinductive material within the first macroscopic feature of the osteoconductive member.

11. The biostructure of claim 10, wherein the cap or formable closure comprises porous material, and the osteoinductive material is accessible to bodily fluids from outside of the biostructure through the pores of the porous material.

12. The biostructure of claim 10, wherein the osteoconductive member comprises a groove for receiving the cap or formable closure.

13. The biostructure of claim 1, wherein the osteoconductive member comprises one or more members of the calcium phosphate family.

14. The biostructure of claim 1, wherein the osteoconductive member comprises beta tricalcium phosphate.

15. The biostructure of claim 1, wherein the osteoconductive member comprises pores having an average pore dimension, and wherein the first macroscopic feature has all dimensions greater than three times the average pore dimension.

16. The biostructure of claim 1, wherein the first macroscopic feature has all dimensions greater than approximately 100 micrometers.

17. The biostructure of claim 1, wherein the first macroscopic feature is tapered.

18. The biostructure of claim 1, wherein the first macroscopic feature includes feature internal dimensions and is connected to an exterior of the biostructure by a channel whose internal cross-section dimensions are smaller than the feature internal dimensions.

19. The biostructure of claim 1, wherein a majority of the osteoinductive material exists in the form of particles having all of their dimensions greater than approximately 100 micrometers.

20. The biostructure of claim 1, wherein the osteoconductive member further comprises a second macroscopic feature which does not contain the material.

21. The biostructure of claim 1, wherein the structure comprises pores having pore sizes between 1 micrometer and 1000 micrometers.

22. The biostructure of claim 1, wherein the osteoconductive member comprises a unitary piece.

23. The biostructure of claim 1, wherein the first macroscopic feature is defined by the union of two osteoconductive members.

24. The biostructure of claim 1, wherein the osteoconductive member comprises two or more pieces suitable to be joined together.

25. The biostructure of claim 1, further comprising an attachment material in contact with the osteoinductive material and the osteoconductive member.

26. The biostructure of claim 25, wherein the attachment material comprises dried gelatin.

27. The biostructure of claim 25, wherein the attachment material comprises gelatin in a gel state.

28. The biostructure of claim 1, wherein the osteoconductive member comprises a matrix material comprising particles partially joined to other particles.

29. The biostructure of claim 28, wherein the particles are joined to other particles by necks having a composition which is substantially the same as the composition of the particles.

30. The biostructure of claim 28, wherein the particles are joined to other particles by necks having a composition which is different from the composition of the particles.

31. The biostructure of claim 28, wherein the particles are partially joined to other particles through a polymer material selected from the group consisting of polylactones; polyamines; polymers and copolymers of trimethylene carbonate with any other monomer; vinyl polymers; acrylic acid copolymers; polyethylene glycols; polyethylenes; Polylactides; Polyglycolides; Epsilon-caprolactone; Polylacatones; Polydioxanones; other Poly(alpha-hydroxy acids); Polyhydroxyalkonates; Polyhydroxybutyrates; Polyhydroxyvalerates; Polycarbonates; Polyacetals; Polyorthoesters; Polyamino acids; Polyphosphoesters; Polyesteramides; Polyfumerates; Polyanhydrides; Polycyanoacrylates; Poloxamers; Polysaccharides; Polyu rethanes; Polyesters; Polyphosphazenes; Polyacetals; Polyalkanoates; Polyurethanes; Poly(lactic acid) (PLA); Poly(L-lactic acid) (PLLA); Poly (DL-lactic acid); Poly-DL-lactide-co-glycolide (PDLGA); Poly(L-lactide-co-glycolide) (PLLGA); Polycaprolactone (PCL); Poly-epsilon-caprolactone; Polycarbonates; Polyglyconates; Polyanhydrides; PLLA-co-GA; PLLA-co-GA 82:18; Poly-DL-lactic acid (PDLLA); PLLA-co-DLLA; PLLA-co-DLLA 50:50; PGA-co-TMC (Maxon B); Polyglycolic acid (PGA); Poly-p-dioxanone (PDS); PDLLA-co-GA; PDLLA-co-GA (85:15); aliphatic polyester elastomeric copolymer; epsilon-caprolactone and glycolide in a mole ratio of from about 35:65 to about 65:35; epsilon-caprolactone and glycolide in a mole ratio of from about 45:55 to about 35:65; epsilon-caprolactone and lactide selected from the group consisting of L-lactide, D-lactide and lactic acid copolymers in a mole ratio of epsilon-caprolactone to lactide of from about 35:65 to about 65:35; Poly(L-lactide and caprolactone in a ratio of about 70:30); poly (DL-lactide and caprolactone in a ratio of about 85:15); poly(DL-lactide and caprolactone and glycolic acid in a ratio of about 80:10:10); poly(DL-lacticde and caprolactone in a ratio of about 75:25); poly(L-lactide and glycolic acid in a ratio of about 85:15); poly(L-lactide and trimethylene carbonate in a ratio of about 70:30); poly(L-lactide and glycolic acid in a ratio of about 75:25); Gelatin; Collagen; Elastin; Alginate; Chitin; Hyaluronic acid; Aliphatic polyesters; Poly(amino acids); Copoly(ether-esters); Polyalkylene oxalates; Polyamides; Poly(iminocarbonates); Polyoxaesters; Polyamidoesters; Polyoxaesters containing amine groups; Poly(anhydrides); and mixtures, copolymers, and terpolymers thereof.

32. The biostructure of claim 28, wherein the matrix material further comprises a blend of at least one active pharmaceutical ingredient and polymer.

33. The biostructure of claim 1, wherein the osteoconductive member has an irregular shape.

34. The biostructure of claim 1, wherein the osteoconductive member is generally cruciform-shaped.

35. The biostructure of claim 1, wherein the osteoconductive member is generally cone, tube, cylinder, box, cruciform, or sphere-shaped.

36. The biostructure of claim 1, wherein the osteoinductive material is selected from the group consisting of fully demineralized bone matrix, partially demineralized bone matrix, osteoinductive bone chip material, cancellous chips, and combinations thereof.

37. The biostructure of claim 1, wherein the osteoinductive material also has osteoconductive characteristics.

38. A biostructure comprising:
an osteoconductive member having a first dimension; and
a coating of material comprising osteoinductive particles on at least a portion of the surface of the osteoconductive member, wherein the coating has a second dimension that is less than the first dimension.

39. The biostructure of claim 38, wherein the osteoconductive member is generally cruciform-shaped.

40. The biostructure of claim 38, wherein the osteoconductive member is generally cone, tube, cylinder, box, cruciform, or sphere-shaped.

41. The biostructure of claim 38, wherein the coating material comprises attachment material.

42. The biostructure of claim 38, wherein the osteoinductive material is selected from the group consisting of fully demineralized bone matrix, partially demineralized bone matrix, osteoinductive bone chip material, cancellous chips, and combinations thereof.

43. The biostructure of claim 38, wherein the osteoconductive member defines at least a first macroscopic feature; and the material is formed within the first macroscopic feature.

44. The biostructure of claim 38, wherein the osteoinductive material also has osteoconductive characteristics.

45. A method of manufacturing a biostructure, the method comprising:
providing an osteoconductive member defining at least a first macroscopic feature; and
depositing a material comprising osteoinductive particles within the first macroscopic feature.

46. The method of claim 45, wherein the material comprises demineralized bone matrix.

47. The method of claim 45, wherein depositing further comprises injecting the material.

48. The method of claim 45, wherein depositing further comprises depositing a material which includes a fat or oil.

49. The method of claim 45, further comprising, after depositing the material, removing a portion of the deposited material by dissolution or rinsing, and then depositing a replacement material.

50. The method of claim 45, further comprising, after depositing, drying the material.

51. The method of claim 45, wherein providing the osteoconductive member comprises providing an osteoconductive member which has both macroscopic features of suitable size for depositing a demineralized bone matrix material and macroscopic features which are too small for depositing the demineralized bone matrix material.

52. The method of claim 45, further comprising a step of joining the osteoconductive member to at least a second osteoconductive member to form the first macroscopic feature.

53. The method of claim 45 further comprising joining the osteoconductive member to at least a second osteoconductive member to form a first macroscopic feature enclosing the material.

54. The method of claim 45, wherein providing the osteoconductive member comprises manufacturing the osteoconductive member in a process comprising three-dimensional printing.

55. The method of claim 45, wherein providing the osteoconductive member comprises manufacturing the osteoconductive member in a process comprising molding.

56. The method of claim 45, wherein providing the osteoconductive member comprises manufacturing the osteoconductive member in a process comprising machining.

57. The method of claim 45, wherein manufacturing the osteoconductive member comprises three dimensional printing onto a powder which comprises a porogen which decomposes into gaseous decomposition products at a certain temperature.

58. The method of claim 45, wherein manufacturing the osteoconductive member comprises three dimensional printing onto a powder which comprises precursors suitable to react to form a desired ceramic substance.

59. The biostructure of claim 45, wherein the osteoinductive material is selected from the group consisting of fully demineralized bone matrix, partially demineralized bone matrix, osteoinductive bone chip material, cancellous chips, and combinations thereof.

60. A biostructure made by the process of claim 45.

61. The biostructure of claim 32, wherein the active pharmaceutical ingredient comprises an antibiotic, an angiogenic factor, an anesthetic, or an osteoinductive substance.

62. The biostructure of claim 32, wherein pores contain the active pharmaceutical ingredient comprises an antibiotic, an angiogenic factor, an anesthetic, or an osteoinductive substance.
